(19) United States
(12) Patent Application Publication
(10) Pub. No.: US 2026/0356409 A1
(43) Pub. Date: Nov. 12, 2026

(54) ENCLOSURE ASSEMBLY FOR A UTILITY CART

(71) Applicant: Apex Brands, Inc., Apex, NC (US)

(72) Inventors: Jesse HIGGINS, Raleigh, NC (US); Robert WARREN, Charlotte, NC (US); Zhihong FU, Cary, NC (US); Kyle ROMANICK, Fuquay-Varina, NC (US)

(21) Appl. No.: 19/137,096

(22) PCT Filed: Dec. 4, 2023
(86) PCT No.: PCT/US2023/082269

§ 371 Date: Jun. 9, 2025

Related U.S. Application Data
(60) Provisional application No. 63/431,845, filed on Dec. 12, 2022.
(60) Provisional application No. 63/468,565, filed on May 24, 2023.

(51) Int. Cl. B62B 3/00; B62B 3/02

(52) U.S. Cl. CPC B62B 3/005; B62B 3/003; B62B 3/004; B62B 3/02; B62B 2202/48

(57) ABSTRACT

A utility cart may include a handle, a top support platform, to which the handle may be operably coupled, a bottom support platform which may be parallel to the top support platform, a plurality of upright support members which may operably couple the bottom support platform to the top support platform, a mobility assembly which may be operably coupled to the bottom support platform, and an enclosure assembly that may be configured to be added to the utility cart between the top support platform and the bottom support platform. The enclosure assembly may be disposed entirely around a perimeter of the bottom support platform to define an enclosed space between the bottom support platform and the top support platform. All fasteners used to operably couple portions of the enclosure assembly to each other may be disposed inside the enclosed space.

### TECHNICAL FIELD

Example embodiments generally relate to utility carts and, in particular, relate to utility carts that are provided with an enclosure assembly for improved utility and versatility.

### BACKGROUND

Utility carts may be commonly used in various settings ranging from dedicated workspaces such as labs, garages, and warehouses or other storage facilities to miscellaneous settings such as in offices, homes, restaurants, and many more. Within the various settings that utility carts may be used, utility carts may also be used for various purposes including but not limited to transporting objects, storing objects, working on objects, displaying objects, and many more. Utility carts may essentially be ubiquitously used in any sort of productive setting involving objects that may be desirable to transport due to their widely applicable and often simple design allowing the cart to be a useful resource in a large amount of use cases.

Existing utility carts may be rather simplistic and outdated in their design considering how they have come to be commonly used today. In this regard, users of utility carts may be using utility carts for more, and different, tasks than in the past. As such, there may be a need to develop an improved design for a utility cart that may be better suited for the increasingly wide variety of tasks for which utility carts may be utilized.

### BRIEF SUMMARY OF SOME EXAMPLES

Some example embodiments may provide for a utility cart. The utility cart may include a handle, a top support platform, to which the handle may be operably coupled, a bottom support platform which may be parallel to the top support platform, a plurality of upright support members which may operably couple the bottom support platform to the top support platform, a mobility assembly which may be operably coupled to the bottom support platform, and an enclosure assembly that may be configured to be added to the utility cart between the top support platform and the bottom support platform. The enclosure assembly may be disposed entirely around a perimeter of the bottom support platform to define an enclosed space between the bottom support platform and the top support platform. All fasteners used to operably couple portions of the enclosure assembly to each other may be disposed inside the enclosed space.

Some example embodiments may provide for an accessory storage assembly for a utility cart. The accessory storage assembly may include a top support platform, a bottom support platform of the utility cart, the bottom support platform may be disposed substantially parallel to the top support platform, and an enclosure assembly which may be configured to be added to the utility cart between the top support platform and the bottom support platform. The enclosure assembly may be disposed entirely around a perimeter of the bottom support platform to define an enclosed space between the bottom support platform and the top support platform. All fasteners used to operably couple portions of the enclosure assembly to each other may be disposed inside the enclosed space.

Some example embodiments may provide for an enclosure assembly for a utility cart. The enclosure assembly may include a first end panel which may be disposed at a first end of the utility cart, a second end panel which may be disposed at a second end of the utility cart opposite from the first end, a front panel assembly which may extend between, and may be operably coupled to, the first and second end panels and may be disposed at a first side of the utility cart, and a back panel which may extend between, and may be operably coupled to, the first and second end panels and may be disposed at a second side of the utility cart opposite from the first side. The first and second end panels may be disposed substantially parallel to each other and substantially perpendicular to the front panel assembly and the back panel. The enclosure assembly may be disposed entirely around a perimeter of the bottom support platform to define an enclosed space between the bottom support platform and the top support platform. All fasteners used to operably couple portions of the enclosure assembly to each other may be disposed inside the enclosed space.

### BRIEF DESCRIPTION OF THE SEVERAL VIEWS OF THE DRAWING(S)

Having thus described some example embodiments in general terms, reference will now be made to the accompanying drawings, which are not necessarily drawn to scale, and wherein:

FIG. 1 illustrates a perspective view of a utility cart according to an example embodiment;

FIG. 2 illustrates a perspective view of the utility cart having an enclosure assembly according to an example embodiment;

FIG. 3 illustrates a perspective view of the enclosure assembly isolated from the cart according to an example embodiment;

FIG. 4 illustrates a perspective view of the enclosure assembly isolated from the cart with a door removed according to an example embodiment;

FIG. 5 illustrates an exploded perspective view of the enclosure assembly isolated from the cart according to an example embodiment;

FIG. 6 illustrates an exploded top view of the enclosure assembly isolated from the cart according to an example embodiment;

FIG. 7 illustrates an isolated perspective view of the front panel assembly of the enclosure assembly in accordance with an example embodiment;

FIG. 8 illustrates an isolated perspective view of an end panel of the enclosure assembly in accordance with an example embodiment;

FIG. 9 illustrates an isolated perspective view of a back panel of the enclosure assembly in accordance with an example embodiment;

FIG. 10 illustrates a close up view of the utility cart and enclosure assembly in accordance with an example embodiment;

FIG. 11 illustrates a perspective view of the enclosure assembly isolated from the cart according to an example embodiment; and

FIG. 12 illustrates a perspective view of the enclosure assembly isolated from the cart according to an example embodiment.

### DETAILED DESCRIPTION

Some example embodiments now will be described more fully hereinafter with reference to the accompanying drawings, in which some, but not all example embodiments are shown. Indeed, the examples described and pictured herein should not be construed as being limiting as to the scope, applicability or configuration of the present disclosure. Rather, these example embodiments are provided so that this disclosure will satisfy applicable legal requirements. Like reference numerals refer to like elements throughout. Furthermore, as used herein, the term “or” is to be interpreted as a logical operator that results in true whenever one or more of its operands are true. As used herein, operable coupling should be understood to relate to direct or indirect connection that, in either case, enables functional interconnection of components that are operably coupled to each other.

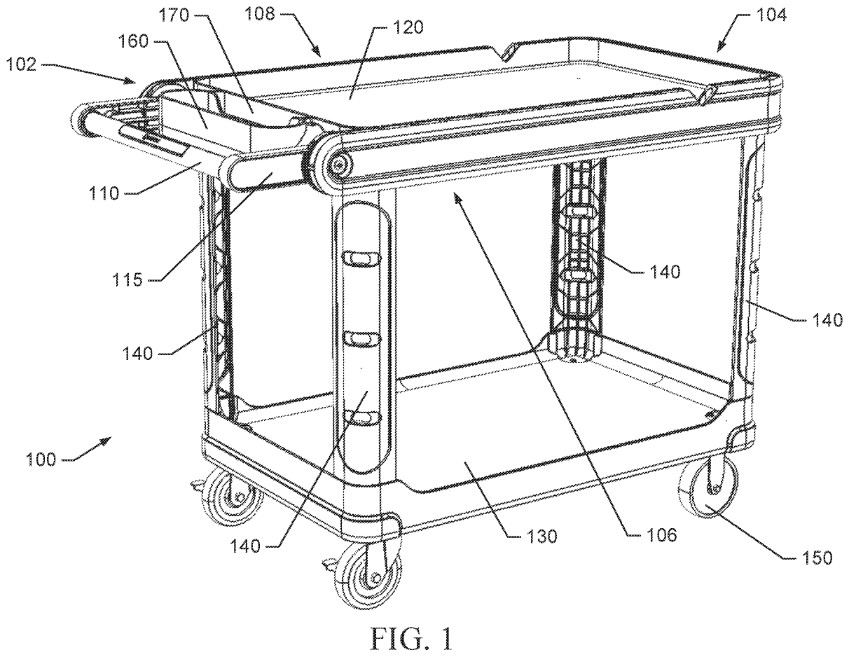

As indicated above, some example embodiments may relate to the provision of a utility cart 100 with an improved design. Referring now to FIG. 1, the utility cart 100 may include a handle 110, a top support platform 120, a bottom support platform 130, upright support members 140 and a mobility assembly. In some embodiments, the handle 110 may be a bar that may be disposed a distance away from the utility cart and may be operably coupled to the utility cart 100 at the top support platform 120 to allow for a user of the utility cart 100 to apply forces to move and steer the utility cart 100. When viewed from above, the utility cart 100 may have a substantially rectangular shape. In this regard, the utility cart 100 may have a first end 102 and a second end 104 and a first side 106 and a second side 108. The first end 102 and the second end 104 may both be equal to each other in length and shorter than each of the first side 106 and second side 108 of the utility cart 100. Thus, in some embodiments, the handle 110 may be operably coupled to the utility cart 100 at the first end 102 of the utility cart 100 proximate to the top support platform 120 so that the handle 110 may be at a comfortable position for the user of the utility cart 100 to interact with. The handle 110 of some example embodiments may also be adjustable. In this regard, the handle 110 may operably couple to the top support platform 120 via at least one extension arm 115. The at least one extension arm 115 may be pivotably operably coupled to the top support platform 120 such that an angle formed between the at least one extension arm 115 and the top support platform 120 may be adjustable. The handle 110 may therefore be fixed in whatever angle or position the user may desire via operable coupling of the at least one extension arm 115 to the top support platform 120.

The top support platform 120 may be disposed a distance above the bottom support platform 130 and supported by the upright support members 140. In some embodiments, there may be four total upright support members 140 supporting the top support platform 120, with one upright support member 140 disposed at each corner of the utility cart 100. It should be noted that in some other embodiments, more or fewer upright support members 140 may be used to support the top support platform 120. The top and bottom support platforms (120, 130) may both be planar surfaces substantially parallel to each other that may be capable of supporting objects thereon. In some cases, the top support platform 120 and the bottom support platform 130 may each include a lip that extends around the entire perimeter of each of the top and bottom support platforms (120, 130) that may help retain objects on respective support platforms by prohibiting the objects from rolling off the utility cart 100.

In some cases, some parts of the utility cart 100 may be formed from structural foam. In this regard, the top and bottom support platforms (120, 130), the handle 110, and some of the accessories described below may be formed from the structural foam. In some embodiments, the structural foam may be a black or gray color which may be a traditional appearance for many structural foams. The use of structural foam to construct the cart 100 may allow the cart 100 to achieve a higher stiffness-to-weight ratio than standard injection molded utility carts while also being more cost effective to produce. In some cases, products formed from structural foam may achieve a weight reduction of approximately 10-20% compared to their injection molded counterparts. However, the utility cart 100 of some embodiments may also include portions that are formed via an injection molding process. In this regard, the upright support members 140 may be injection molded from impact modified polypropylene, which may allow for the upright support members 140 to be colored without impacting the structural integrity of the cart 100.

Operably coupled to an underside of the bottom support platform 130 may be the mobility assembly, which may include a plurality of wheels 150. In some cases, the utility cart may include a total of four wheels 150, though more or less may also be used in other cases. The wheels 150 may be disposed at each of the four corners of the utility cart 100 shown in FIG. 1 in order to maximize the balance of the utility cart 100 and limit instability of the cart 100. In an example embodiment, all of the wheels 150 on the utility cart may be caster wheels. In such cases, all of the wheels 150 may be capable of turning 360° to allow the utility cart 100 to travel in any direction regardless of the orientation of the cart 100. In some other cases, the two wheels 150 disposed at the first end 102 may be caster wheels and the two wheels disposed at the second end 104 may be fixed wheels. In this regard, the utility cart 100 may be capable of being steered from the handle 110 at the first end 102 responsive to the caster wheels being capable of turning in any direction. In another example embodiment, the two wheels 150 disposed at the second end 104 may be caster wheels and the two wheels disposed at the first end 102 may be fixed wheels. In this regard, the utility cart 100 may be capable of being steered at the second end 104 via the handle 110 responsive to the caster wheels being capable of turning in any direction. In some cases, the wheels 150 may be fitted with a parking brake on each wheel 150. Each parking brake may immobilize the respective wheel 150 to which it may be operably coupled. With the parking brake for each wheel 150 engaged, the utility cart 100 may be held in place and may not roll anywhere. This feature may be useful when work may be done on the utility cart 100 itself, or in other cases where the cart 100 may roll away from the user unintentionally.

In some embodiments, the utility cart 100 may further include a storage pocket 160. The storage pocket 160 may be disposed between the handle 110 and the top support platform 120 in order to be easily accessible by the user of the utility cart 100. The storage pocket 160 may be capable of storing a number of objects of many different varieties. For example, the storage pocket 160 may store hand tools, writing utensils, or other miscellaneous objects of similar sizes. The storage pocket 160 may be disposed a distance away from the handle 110 due to the extension arms 115 extending away from the top support platform 120. In some cases, the distance between the handle 110 and the storage pocket 160 may vary since the position of the handle 110 may be adjustable. The storage pocket 160 may be disposed between two extension arms 115 of the handle 110. In this regard, there may be excess space on either longitudinal end of the storage pocket 160 between the storage pocket 160 and the extension arms 115. This space may provide some buffer between the storage pocket 160 and the extension arms 115 so that any objects stored in the storage pocket 160 do not interfere with the adjustment of the position of the handle 110 via the extension arms 115. In some cases, the space between the longitudinal ends of the storage pocket 160 and the extension arms 115 may account for approximately 10% of the total length of the handle 110, or a total distance between the two extension arms 115. In other words, the storage pocket 160 may have a length that is approximately 90% of the total length of the handle 110, or the total distance between the two extension arms 115.

The utility cart 100 may further include a technology receptacle 170 disposed between the storage pocket 160 and the top support platform 120. The technology receptacle 170 may be designed to support a personal electronic device, such as a mobile phone device or a tablet device, in a position where the personal electronic device may be used or viewed by the user of the utility cart 100. As such, the technology receptacle 170 may keep the personal electronic device separate from the storage of other objects within the storage pocket 160 to prevent any interference of the personal electronic device due to objects in the storage pocket 160. In contrast with the technology receptacle 170, the storage pocket 160 may be designed to hold a variety of tools, materials and objects that the user may want to keep separate or more accessible than objects in either of the top or bottom support platforms (120, 130). In an example embodiment, the distance between the storage pocket 160 and the handle 110 may not exceed a width of the storage pocket 160 measured between a shared wall with the technology receptacle 170 and a front wall of the storage pocket 160 closest to the handle 110. In some other cases, the distance between the storage pocket 160 and the handle 110 may be within a range of approximately 50-75% of the width of the storage pocket 160. This may ensure that there is sufficient space between the storage pocket 160 and the handle so that they may not interfere with each other as the user may interact with the handle 110 or the storage pocket 160. In an example embodiment, the technology receptacle 170 may have an overall length that is approximately 75% of the length of the storage pocket 160. In other words, the overall length of the technology receptacle 170 may be approximately 50-60% of the total length of the handle 110, or the total distance between the two extension arms 115. In an example embodiment, the top support platform 120 may be wider than the handle 110 is long. In other words, the length of the handle 110 may be approximately 90% of a width of the top support platform 120. Similarly, the length of the technology receptacle 170 may be approximately 40-50% of the width of the top support platform 120.

Thus, the technology receptacle 170 may hold the user's personal electronic device therein. For example, in a scenario where the utility cart 100 may be used for order fulfillment situations, the personal electronic device may contain a list of items needed to fulfill a particular order. Thus, the user of the cart 100 may need to easily see the screen of the personal electronic device being supported in the technology receptacle 170. In this regard, the technology receptacle 170 may provide stability of the personal electronic device disposed in the technology receptacle 170 so that the movement of the utility cart 100 does not knock over the personal electronic device. Additionally, the technology receptacle 170 may be designed to provide the user with an ergonomic view of the display of the personal electronic device so that the user of the utility cart 100 does not need to strain to view the device.

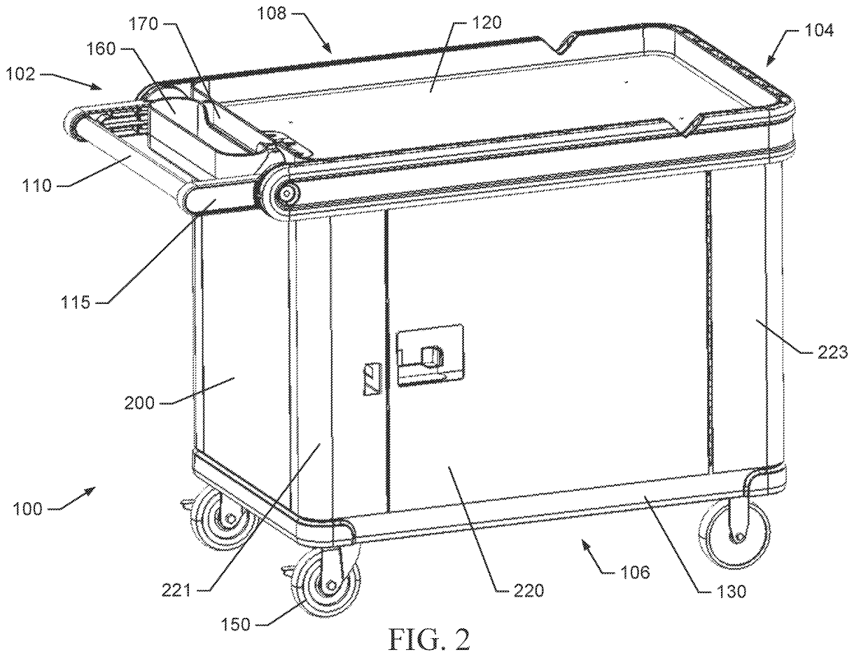

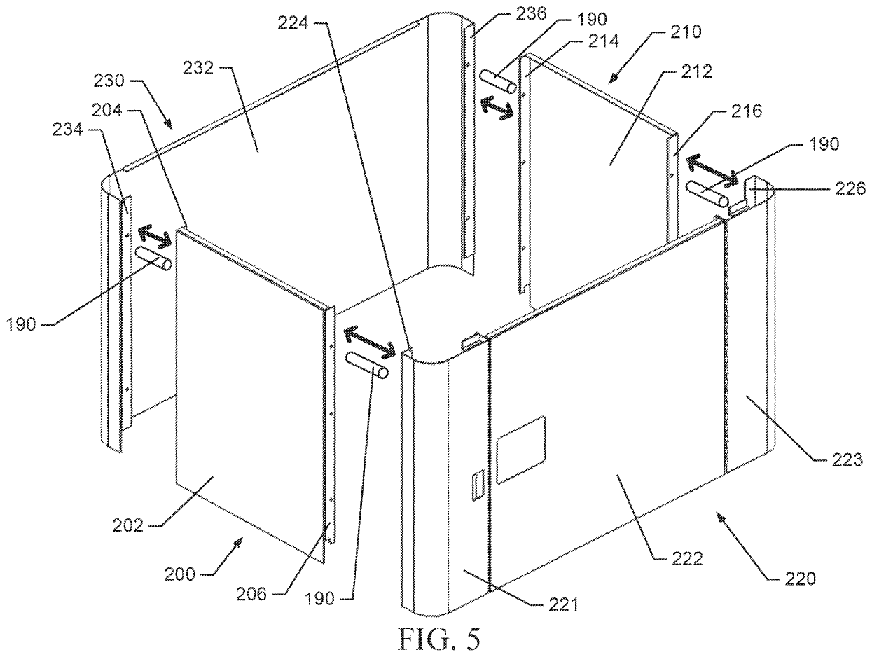

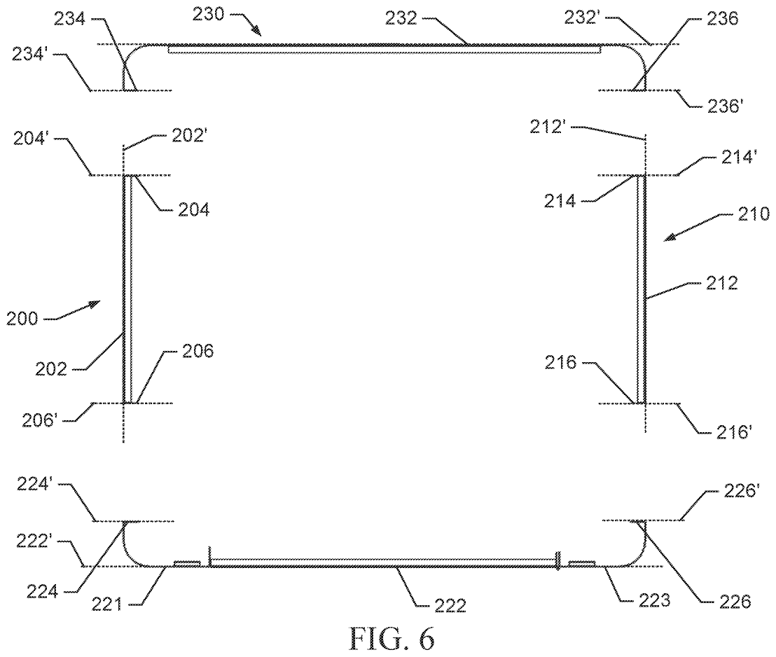

FIG. 2 illustrates a perspective view of the utility cart 100 having an enclosure assembly 180 in accordance with an example embodiment. In this regard, the enclosure assembly 180 may be added to the utility cart 100 between the top support platform 120 and the bottom support platform 130, and around an exterior surface of the upright support members 140. Thus, the enclosure assembly 180 may be disposed entirely around a perimeter of the bottom support platform 130 to define an enclosed space between the bottom support platform 130 and the top support platform 120. The upright support members 140 may accordingly be disposed within the enclosed space when the enclosure assembly 180 is assembled on the utility cart 100. In this regard, the enclosure assembly 180 may be added to, and removed from, the utility cart 100 by the user depending on the needs of the user. In some cases, a plurality of fasteners 190 may be used to operably couple the individual components of the enclosure assembly 180 together on the utility cart 100. In an example embodiment, all fasteners 190 used to operably couple the enclosure assembly 180 to the utility cart 100 may be disposed inside the enclosed space defined by the enclosure assembly 180. This may improve the security of the enclosure assembly 180 by prohibiting components of the enclosure assembly 180 from being removed from the utility cart 100 without first having access into the enclosed space. Further, the components of the enclosure assembly 180 may be operably coupled to each other via the fasteners 190, but not to the utility cart 100 itself. This may allow the enclosure assembly 180 to completely enclose the upright support members 140 of the utility cart 100. In fact, in an example embodiment, the enclosure assembly 180 may be assembled separate from the utility cart 100 and accordingly installed onto the utility cart 100 as one unitary component. In this regard, the top support platform 120 may be removable from the plurality of upright support members 140. Thus, in order to install the enclosure assembly 180 onto the cart 100 in one piece, the user may simply remove the top support platform 120, slide the enclosure assembly 180 into place around each of the upright support members 140, and then reinstall the top support platform 120 thereon after the enclosure assembly 180 has been installed. The fasteners 190 will be discussed in further detail below in reference to FIGS. 5 and 6.

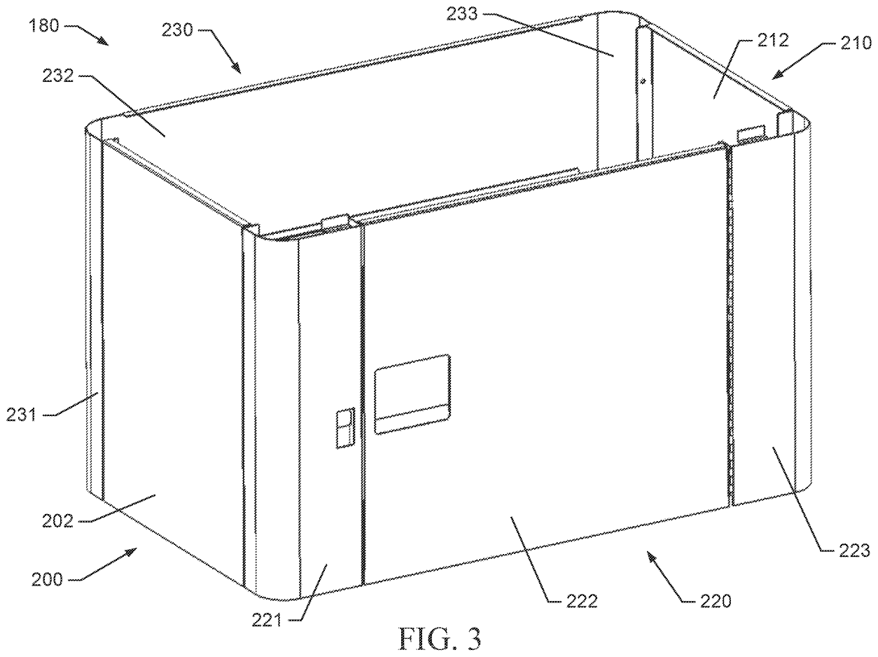

As shown in FIGS. 3-9, the enclosure assembly 180 may include a first end panel 200, a second end panel 210, a front panel assembly 220, and a back panel 230. The first end panel 200 may be disposed at the first end 102 of the utility cart 100 while the second end panel 210 may be disposed at the second end 104 of the utility cart 100, opposite from the first end 102. The front panel assembly 220 may extend between, and be operably coupled to, the first and second end panels (200, 210) and may be disposed at the first side 106 of the utility cart 100. On the other hand, the back panel 230 may extend between, and be operably coupled to, the first and second end panels (200, 210) and may be disposed at the second side 108 of the utility cart 100, opposite from the first side 106. The enclosure assembly 180 may thus be assemblable and dissassemblable on a component-wise basis. In other words, the user may add the enclosure assembly 180 to the utility cart 100 themselves by installing the enclosure assembly 180 one component at a time. In an example embodiment, the back panel 230 may be installed onto the utility cart 100 first, followed by each of the first and second end panels (200, 210) not in any particular order. Then, the front panel assembly 220 may be installed to complete the perimeter of the enclosure assembly 180. Each of the back panel 230, the first and second end panels (200, 210) and the front panel assembly 220 may extend upright between the top and bottom support platforms (120, 130) and may form an angle substantially equal to 90° with each of the top and bottom support platforms (120, 130). In some cases, the enclosure assembly 180 may be made from 18 gauge sheet metal, but other materials with similar physical properties may be used in other cases. In this regard, the enclosure assembly 180 may act as armor for the utility cart 100 since the enclosure assembly 180 may protect the enclosed space from other objects or people that may attempt to enter the enclosed space. In some cases, the enclosure assembly 180 may be magnetic. In this regard, the user may utilize magnets to keep objects secured to the enclosure assembly 180 either in the enclosed space of the enclosure assembly 180 or on an exterior surface of the enclosure assembly 180. In an example embodiment, the enclosure assembly 180 may be customized with different powder coating options or different paint options depending on the desires and needs of the user.

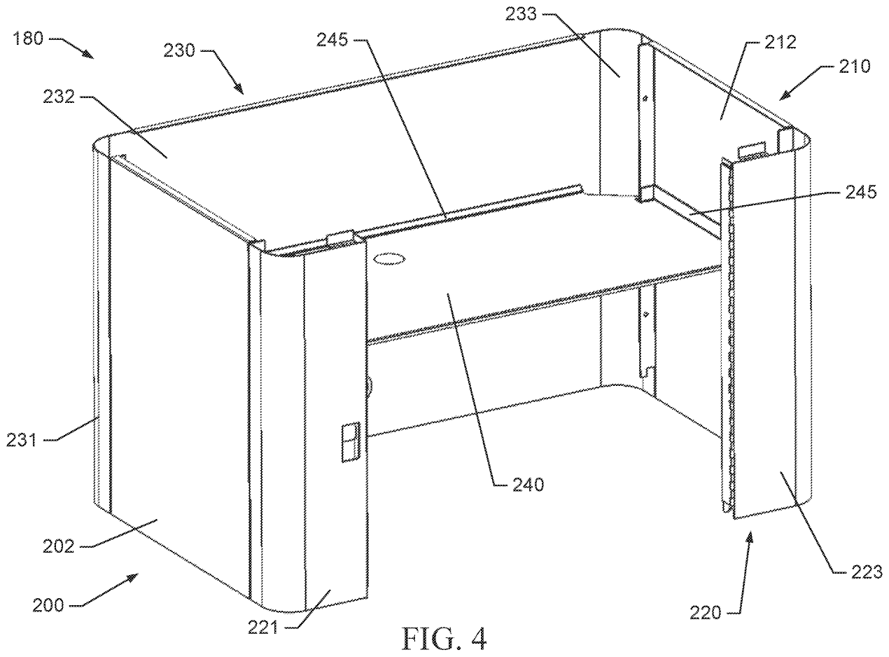

As shown in FIG. 4, in some cases, the enclosure assembly 180 may further include a shelf 240 which may be disposed parallel to the top support platform 120 and the bottom support platform 130 within the enclosed space. The shelf 240 may be supported by a support lip 245 disposed at respective interior facing surfaces of the first end panel 200, the second end panel 210, and the back panel 230. In this regard, the shelf 240 may be supported by the support lip 245 at three edges of the shelf 240. The fourth edge of the shelf 240 may face the front panel assembly 220 and may not reach entirely to the front panel assembly 220 in some cases. In fact, in an example embodiment, the shelf 240 may extend to cover approximately 70% of a depth of the enclosed space, which may be measured as the distance between the back panel 230 and the front panel assembly 220. In other words, a depth of the shelf 240 measured from a front edge of the shelf 240 to the back panel 230 may be approximately 30% shorter than the depth of the enclosed space. This may allow the user to reach/access objects stored both on top of the shelf 240 and under the shelf 240 on the bottom support platform 130 from the front panel assembly 220 when a door in the front panel assembly 220 may be open. In some cases, the shelf 240 may be removable from the remainder of the enclosure assembly 180 such that the enclosure assembly 180 may be used both with and without the shelf 240. In this regard, the shelf 240 may be held in place on the support lip 245 simply by gravity. Thus, the weight of the shelf 240 may be substantial enough to operably couple the shelf 240 to the support lip 245 while the utility cart 100 moves around and is used accordingly. The support lip 245 of some example embodiments may include some sound deadening, insulation, and/or vibration reduction material to reduce the propagation of noise and vibrations that may arise due to the shelf 240 vibrating with the support lip 245 as the utility cart 100 moves and is used accordingly. In some cases, two of the corners of the shelf 240 that may be disposed between the three edges of the shelf 240 that may operably couple to the support lip 245 may be cut off diagonally. This may allow the shelf 240 to bypass the upright support members 140 that may be disposed in corner portions of the back panel 230 and not leave any gaps between the shelf 240 and the upright support members 140 that any object could fall down between. The shelf 240 of some embodiments may thus be a drop-in panel that may self-locate by utilizing the support lip 245.

Referring particularly now to FIGS. 5-9, the enclosure assembly 180 is shown in an exploded view that may highlight each of the components of the enclosure assembly 180 and how they interact with one another. As such, the first end panel 200 may include a first outer face 202, a first attachment flange 204, and a second attachment flange 206, and the second end panel 210 may include a second outer face 212, a third attachment flange 214, and a fourth attachment flange 216. In an example embodiment, the first, second, third and fourth attachment flanges (204, 206, 214, 216) may all be disposed inside the enclosed space. In other words, on one hand, the first and second outer faces (202, 212) may define the planar portions of the first and second end panels (200, 210) that may fill the space between consecutive upright support members 140 at the first and second ends (102, 104) of the utility cart 100. On the other hand, the first, second, third and fourth attachment flanges (204, 206, 214, 216) may be disposed at opposing edges of, and may extend into the enclosed space from, their respective one of the first and second end panels (200, 210). Furthermore, in some cases, each of the first, second, third and fourth attachment flanges (204, 206, 214, 216), like the first and second outer faces (202, 212), may extend from the top support platform 120 to the bottom support platform 130 continuously.

The first outer face 202 and the second outer face 212 of some embodiments may be disposed in respective first and second end planes (202′, 212′) accordingly, while the first, second, third and fourth attachment flanges (204, 206, 214, 216) may be disposed in respective first, second, third and fourth attachment planes (204′, 206′, 214′, 216′), respectively. In some cases, the first, second, third and fourth attachment planes (204′, 206′, 214′, 216′) may be substantially perpendicular to the first and second end planes (202′, 212′). Additionally, the first, second, third and fourth attachment planes (204′, 206′, 214′, 216′) may all be parallel with each other, and the first and second end planes (202′, 212′) may be parallel with each other as well.

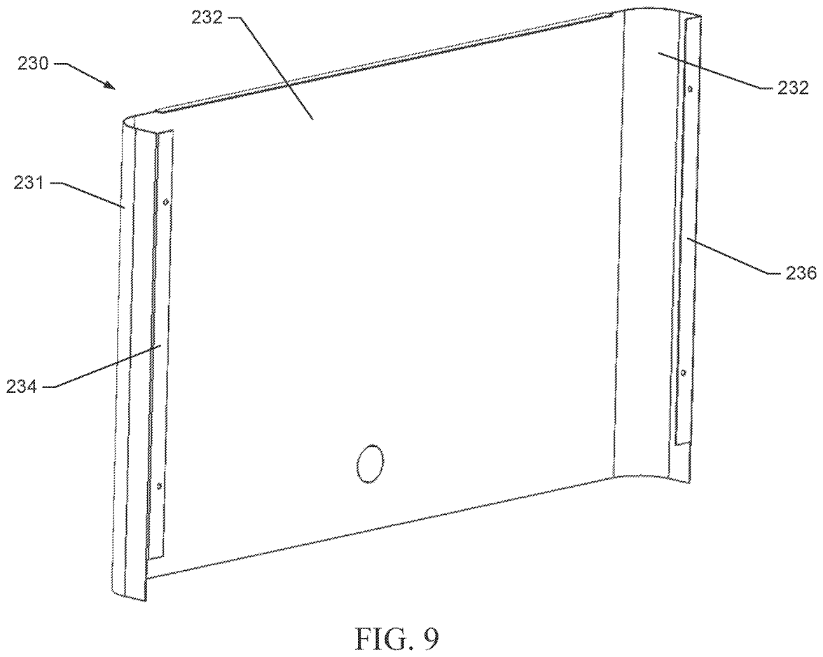

Similarly, the back panel 230, as shown in FIG. 9, may include a third outer face 232, a fifth attachment flange 234, and a sixth attachment flange 236. However, the back panel 230 may also include first and second corner portions (231, 233) which may be configured to extend around an exterior surface of the upright support members 140 proximate to the first and second end panels (200, 210). Thus, the first and second corner portions (231, 233) may include the fifth attachment flange 234 and sixth attachment flange 236, respectively. The third outer face 232 may be disposed in a first side plane 232′ and the fifth and sixth attachment flanges (234, 236) may be disposed in respective fifth and sixth attachment planes (234′, 236′) that may be parallel to the first and third attachment planes (204′, 214′), respectively. The fifth and sixth attachment planes (234′, 236′) may also be parallel to the first side plane 232′. In this regard, the first and second corner portions (231, 233) may engage the upright support members 140 to align the back panel 230 in proper orientation with the rest of the utility cart 100 so that the third outer face 232 may extend to cover the space between consecutive upright support members 140 at the second side 108 of the utility cart 100.

Thus, as shown in FIGS. 5-9, the first and second end panels (200, 210) may be operably coupled to the back panel 230 via a first fastener 190 that may operably couple the first and fifth attachment flanges (204, 234) to each other and a second fastener 190 that may operably couple the third and sixth attachment flanges (214, 236) to each other, respectively. In this regard, the first fastener 190 may be disposed perpendicular through the first and fifth attachment planes (204′, 234′) and the second fastener 190 may be disposed perpendicular through the third and sixth attachment planes (214′, 236′). In some cases, a plurality of fasteners 190 may be used to operably couple each of the first and second end panels (200, 210) to the back panel 230 via respective attachment flanges.

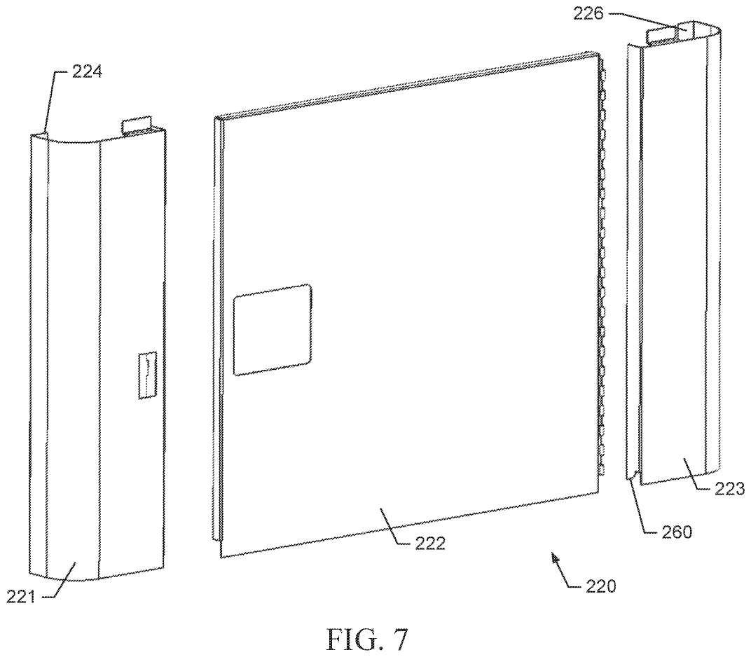

FIG. 7 depicts an isolated perspective view of the front panel assembly 220. In some cases, the front panel assembly 220 may include a third corner portion 221, a fourth corner portion 223, and at least one door 222 hingedly operably coupled to one of the third corner portion 221 and the fourth corner portion 223. Similar to the first and second corner portions (231, 233) of the back panel 230, the third and fourth corner portions (221, 223) may include seventh and eighth attachment flanges (224, 226), respectively. In some cases, the at least one door 222 may be disposed in a second side plane 222′ and the seventh and eighth attachment flanges (224, 226) may be disposed in respective seventh and eighth attachment planes (224′, 226′). Again, similar to the back panel 230, the seventh and eighth attachment planes (224′, 226′) may be parallel to the second and fourth attachment planes (206′, 216′), respectively. Thus, the first and second end panels (200, 210) may be operably coupled to the front panel assembly 220 via a third fastener 190 that may operably couples the second and seventh attachment flanges (206, 224) to each other and a fourth fastener 190 that may operably couple the fourth and eighth attachment flanges (216, 226) to each other, respectively. Therefore, the third fastener 190 may be disposed perpendicular through the second and seventh attachment planes (206′, 224′) and the fourth fastener may be disposed perpendicular through the fourth and eighth attachment planes (216′, 226′).

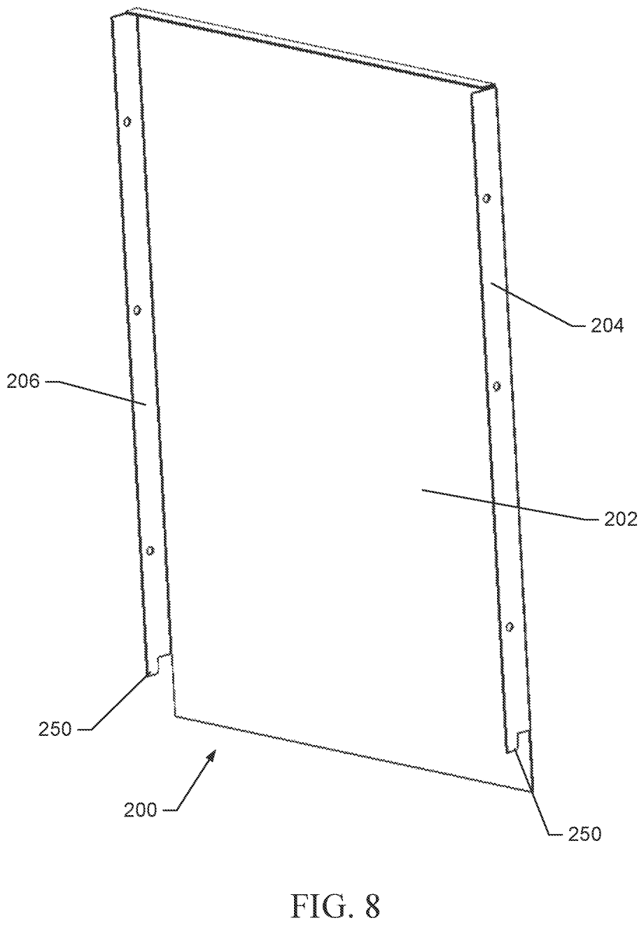

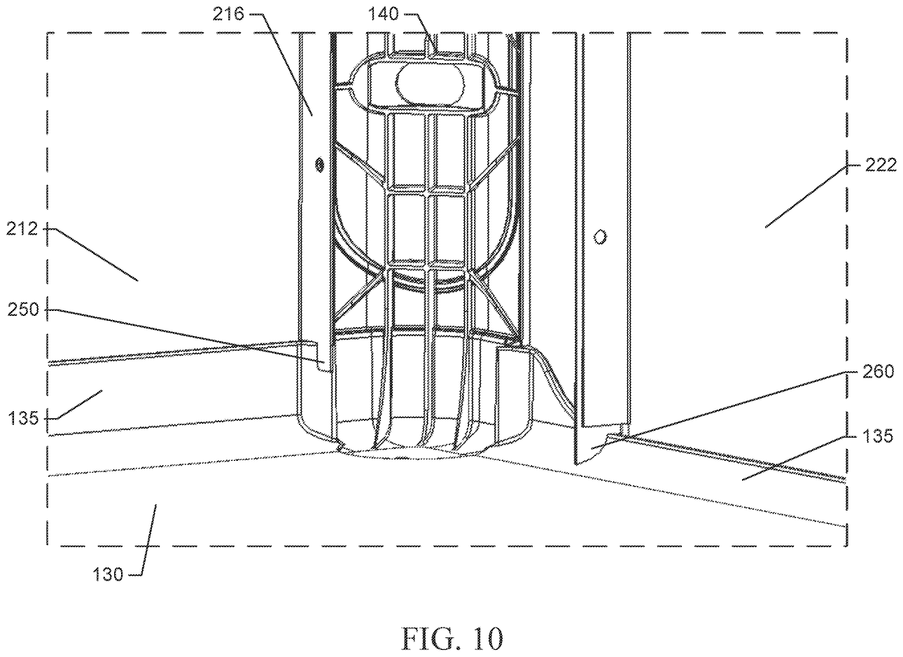

As shown in FIGS. 8 and 10, the first, second, third and fourth attachment flanges (204, 206, 214, 216) of the first and second end panels (200, 210) may include an end hook 250 disposed proximate to the bottom support platform 130. In some cases, the end hook 250 may be configured to engage a lip 135 of the bottom support platform 130 at the first and second ends (102, 104) of the cart 100 to operably couple the first and second end panels (200, 210) to the bottom support platform 130. In this regard, the first and second end panels (200, 210) may be inserted onto the utility cart 100 by first placing the lower edge of the first and second outer faces (202, 212) so that it may contact an exterior surface of the lip 135 along the first or second end (102, 104) of the utility cart 100. Then, an upper edge of the first and second end panels (200, 210) may be pivoted toward the top support platform 120 such that the end hook 250 proximate the lip 135 of the bottom support platform 130 extends from outside the lip 135, across the top of the lip 135, and into the bottom support platform 130. Finally, the first and second end panels (200, 210) may be lowered until the end hooks 250 engage the lip 135 of the bottom support platform 130 to operably couple the first and second end panels (200, 210) to the lip 135 of the bottom support platform 130 as shown in FIG. 10. Also shown in FIG. 10, and similar to the first and second end panels (200, 210), the front panel assembly 220 may include a side hook 260 at each of the seventh and eighth attachment flanges (224, 226) disposed proximate to the bottom support platform 130. In this regard, the side hook 260 disposed at the seventh attachment flange 224 may engage the lip 135 at the first side 106 of the cart 100 to operably couple the third corner portion 221 to the bottom support platform 130, and the side hook 260 disposed at the eighth attachment flange 226 may engage the lip 135 at the first side 106 of the cart 100 to operably couple the fourth corner portion 223 to the bottom support platform 130 to support the weight of the front panel assembly 220, and align the front panel assembly 220 with the first and second end panels (200, 210) accordingly.

In some cases, the door 222 may be hingedly operably coupled to the third corner portion 221 or the fourth corner portion 223. Thus, the door 222 may be operable between a first state in which the door 222 is closed and the enclosure assembly 180 entirely encloses the enclosed space, and a second state in which the door 222 is open and the enclosed space is accessible via the open door 222. In some other cases, the door 222 may be slidably operably coupled to the third corner portion 221 or the fourth corner portion 223. In this regard, the door 222 may still be operable between the first and second states, but the door 222 may slide between the first and second states rather than pivot between the first and second states. In an example embodiment, the door 222 may be lockable to securely store objects inside of the enclosure assembly 180 in the enclosed space therein.

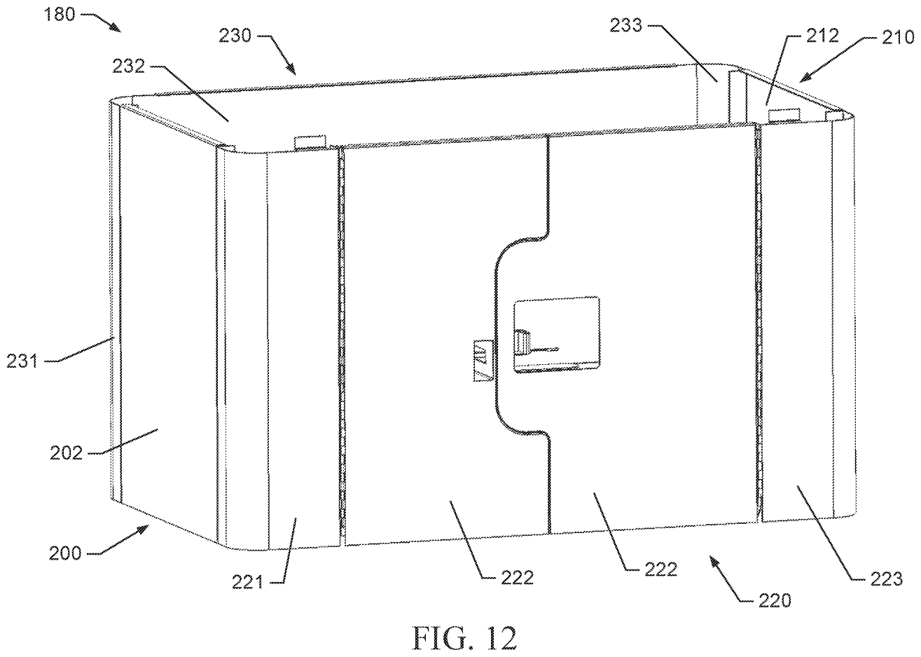

The enclosure assembly 180 shown in the embodiment of FIGS. 2-11 may include a singular door 222 that may provide access to the enclosed space of the enclosure assembly 180. However, as shown in FIG. 12, in some cases there may be two or more doors 222. In such cases, the doors 222 may be double doors 222 where both doors 222 may be disposed at a same side (106 or 108) of the utility cart 100. The double door 222 design may allow for more side clearance when opening the doors 222 due to the reduced swing length of each door 222. In some other cases, the two or more doors 222 may be disposed at different sides (106, 108) of the utility cart 100 which may allow the user to access the enclosed space from various entry positions.

In some cases, the door 222 may be lockable via a locking assembly. The locking assembly may include a padlock housing and a hook. In some cases, the padlock housing may be disposed in the third corner portion 221 or the fourth corner portion 223, and the hook may be disposed in the door 222. With the door 222 open, a padlock may be inserted into the padlock housing in an unlocked state. In order to close and lock the door 222, the user may first slide the hook to an open position towards the hinges of the door 222, and then proceed to close the door 222 with the hook remaining in the open position. Responsive to the door 222 being closed, the user may slide the hook back, away from the hinges of the door 222, to a closed position. In the closed position, the hook may prohibit the door 222 from being opened by interfering with the padlock housing. With the hook in the closed position, the padlock disposed within the padlock housing may be locked. When the padlock is locked, it may interfere with the travel of the hook and thus not allow the door 222 to be opened without unlocking the padlock to release the hook. In an example embodiment, the padlock may be any number of well-known padlocks available to purchase. In some cases, the padlock may be alternatingly locked and unlocked via a key, a numeric key code, or any other form of locking mechanism.

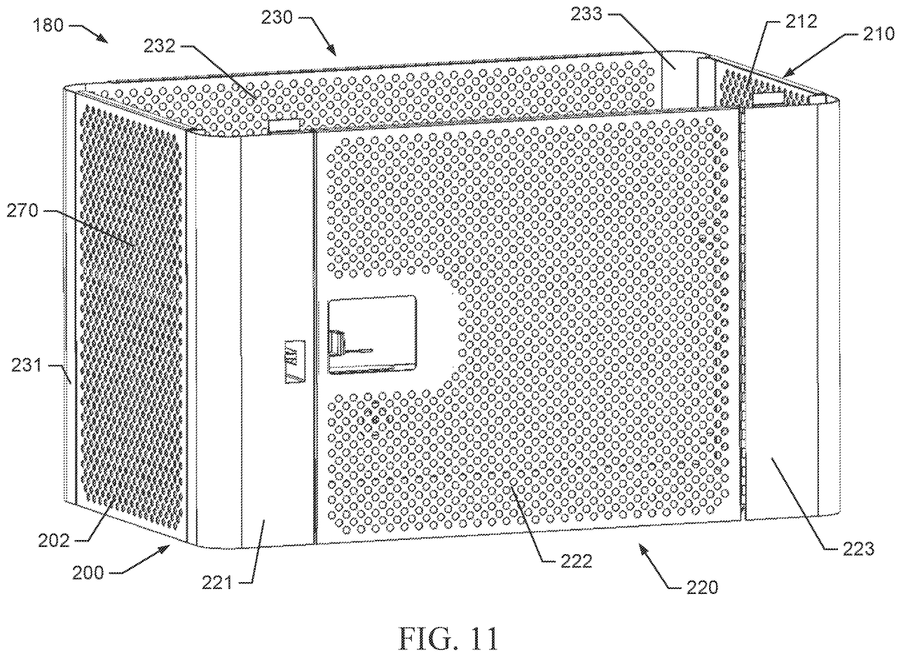

FIG. 11 depicts a further example embodiment of the enclosure assembly 180 accordingly. In the embodiment of FIG. 11, the enclosure assembly 180 may further include a pattern comprising a plurality of holes 270 that may allow visibility into the enclosed space. In this regard, the enclosure assembly 180 may be made of material comprising a mesh pattern of holes 270 to provide some transparency through the enclosure assembly 180. In this regard, the mesh enclosure assembly 180 may be assembled and operated in the same manner as the opaque enclosure assembly 180 shown and described above in reference to FIGS. 2-10 and 12, and may include the same types of doors 222 described above with respect to the opaque enclosure assembly 180. Thus, the mesh enclosure assembly 180 may still securely store items in the bottom support platform 130, but the mesh enclosure assembly 180 may allow the user to see the contents of the enclosed space through the enclosure assembly 180. In some embodiments, the mesh enclosure assembly 180 may function both as an enclosure assembly 180 and also as an additional storage solution. In this regard, the user may insert fasteners into the enclosure assembly 180 panels in order to secure objects, such as hand tools, directly to the enclosure assembly 180 panels via the holes 270. The objects may be secured directly to the panels on the interior or the exterior of the enclosure assembly 180. Securing objects directly to the panels of the enclosure assembly 180 may help keep the bottom support platform 130 less cluttered by removing some objects that would typically be taking up space on the bottom support platform 130 and securing them to the panels instead so that the bottom support platform 130 may be used to store larger objects.

In some example embodiments, the utility cart 100 may include an accessory storage assembly 280. In this regard, the accessory storage assembly 280 may include the top support platform 120, the bottom support platform 130 which may be disposed substantially parallel to the top support platform 120, and the enclosure assembly 180 which may be configured to be added to the utility cart 100 between the top support platform 120 and the bottom support platform 130. The enclosure assembly 180 may be disposed entirely around the perimeter of the bottom support platform 130 to define the enclosed space between the bottom support platform 130 and the top support platform 120. All fasteners 190 used to operably couple the enclosure assembly 180 to the utility cart 100 may be disposed inside the enclosed space.

Some example embodiments may provide for a utility cart. The utility cart may include a handle, a top support platform, to which the handle may be operably coupled, a bottom support platform which may be parallel to the top support platform, a plurality of upright support members which may operably couple the bottom support platform to the top support platform, a mobility assembly which may be operably coupled to the bottom support platform, and an enclosure assembly that may be configured to be added to the utility cart between the top support platform and the bottom support platform. The enclosure assembly may be disposed entirely around a perimeter of the bottom support platform to define an enclosed space between the bottom support platform and the top support platform. All fasteners used to operably couple portions of the enclosure assembly to each other may be disposed inside the enclosed space.

The utility cart of some embodiments may include additional, optional features, and/or the features described above may be modified or augmented. Some examples of modifications, optional features and augmentations are described below. It should be appreciated that the modifications, optional features and augmentations listed below may each be added alone, or they may be added cumulatively in any desirable combination. For example, in some embodiments, the enclosure assembly may include a first end panel which may be disposed at a first end of the utility cart, a second end panel which may be disposed at a second end of the utility cart opposite from the first end, a front panel assembly which may extend between, and may be operably coupled to, the first and second end panels and may be disposed at a first side of the utility cart, and a back panel which may extend between, and may be operably coupled to, the first and second end panels and may be disposed at a second side of the utility cart opposite from the first side. In some cases, the first end panel may include a first outer face, a first attachment flange, and a second attachment flange. In an example embodiment, the second end panel may include a second outer face, a third attachment flange, and a fourth attachment flange. In some cases, the first, second, third and fourth attachment flanges may be disposed inside the enclosed space. In an example embodiment, the first, second, third and fourth attachment flanges may extend from the top support platform to the bottom support platform. In some cases, the first, second, third, and fourth attachment flanges of the first and second end panels may include a hook which may be disposed proximate to the bottom support platform. In an example embodiment, the hook may be configured to engage a lip of the bottom support platform to operably couple the first and second end panels to the bottom support platform. In some cases, the first outer face and the second outer face may be disposed in respective first and second end planes. In an example embodiment, the first, second, third and fourth attachment flanges may be disposed in respective first, second, third and fourth attachment planes. In some cases, the first, second, third and fourth attachment planes may be substantially perpendicular to the first and second end planes. In an example embodiment, the back panel may include a third outer face, a fifth attachment flange, and a sixth attachment flange. In some cases, the third outer face may be disposed in a first side plane and the fifth and sixth attachment flanges may be disposed in respective fifth and sixth attachment planes that may be parallel to the first and third attachment planes, respectively. In an example embodiment, the back panel may further include a first corner portion, and a second corner portion. In some cases, the first and second corner portions may be shaped to extend around an exterior surface of respective ones of the plurality of upright support members. In an example embodiment, the fifth and sixth attachment flanges may be disposed at the first and second corner portions, respectively. In some cases, the fifth and sixth attachment planes may be parallel to the first side plane. In an example embodiment, the first and second end panels may be operably coupled to the back panel via a first fastener that may operably couple the first and fifth attachment flanges to each other and a second fastener that may operably couple the third and sixth attachment flanges to each other, respectively. In some cases, the first fastener may be disposed perpendicular to the first and fifth attachment planes and the second fastener may be disposed perpendicular to the third and sixth attachment planes. In an example embodiment, the front panel assembly may include a third corner portion, a fourth corner portion, and at least one door that may be hingedly operably coupled to one of the first corner portion and the second corner portion. In some cases, the third and fourth corner portions may include seventh and eighth attachment flanges, respectively. In an example embodiment, the at least one door may be disposed in a second side plane and the seventh and eighth attachment flanges may be disposed in respective seventh and eighth attachment planes. In some cases, the seventh and eighth attachment planes may be parallel to the second and fourth attachment planes, respectively. In an example embodiment, the first and second end panels may be operably coupled to the front panel assembly via a third fastener that may operably couple the second and seventh attachment flanges to each other and a fourth fastener that may operably couple the fourth and eighth attachment flanges to each other, respectively. In some cases, the third fastener may be disposed perpendicular to the second and seventh attachment planes and the fourth fastener may be disposed perpendicular to the fourth and eighth attachment planes. In an example embodiment, the seventh and eighth attachment flanges of the front panel assembly may each include a hook which may be disposed proximate to the bottom support platform. In some cases, the hook may be configured to engage a lip of the bottom support platform to operably couple the third and fourth corner portions to the bottom support platform. In an example embodiment, the enclosure assembly may further include a shelf disposed parallel to the top support platform and the bottom support platform within the enclosed space. In some cases, the shelf may be supported by a support lip disposed at the first end panel, the second end panel, and the back panel. In an example embodiment, the enclosure assembly may further include a pattern comprising a plurality of holes that may allow visibility into the space between the bottom storage platform and the top support platform. In some cases, the enclosure assembly may be opaque.

Some example embodiments may provide for an accessory storage assembly for a utility cart. The accessory storage assembly may include a top support platform, a bottom support platform of the utility cart, the bottom support platform may be disposed substantially parallel to the top support platform, and an enclosure assembly which may be configured to be added to the utility cart between the top support platform and the bottom support platform. The enclosure assembly may be disposed entirely around a perimeter of the bottom support platform to define an enclosed space between the bottom support platform and the top support platform. All fasteners used to operably couple portions of the enclosure assembly to each other may be disposed inside the enclosed space.

Some example embodiments may provide for an enclosure assembly for a utility cart. The enclosure assembly may include a first end panel which may be disposed at a first end of the utility cart, a second end panel which may be disposed at a second end of the utility cart opposite from the first end, a front panel assembly which may extend between, and may be operably coupled to, the first and second end panels and may be disposed at a first side of the utility cart, and a back panel which may extend between, and may be operably coupled to, the first and second end panels and may be disposed at a second side of the utility cart opposite from the first side. The first and second end panels may be disposed substantially parallel to each other and substantially perpendicular to the front panel assembly and the back panel. The enclosure assembly may be disposed entirely around a perimeter of the bottom support platform to define an enclosed space between the bottom support platform and the top support platform. All fasteners used to operably couple portions of the enclosure assembly to each other may be disposed inside the enclosed space.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe exemplary embodiments in the context of certain exemplary combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. In cases where advantages, benefits or solutions to problems are described herein, it should be appreciated that such advantages, benefits and/or solutions may be applicable to some example embodiments, but not necessarily all example embodiments. Thus, any advantages, benefits or solutions described herein should not be thought of as being critical, required or essential to all embodiments or to that which is claimed herein. Although specific terms are employed herein, they are used in a generic and descriptive sense only and not for purposes of limitation.

1. A utility cart comprising:
a handle;
a top support platform, to which the handle is operably coupled;
a bottom support platform disposed parallel to the top support platform;
a plurality of upright support members operably coupling the bottom support platform to the top support platform;
a mobility assembly operably coupled to the bottom support platform; and
an enclosure assembly configured to be added to the utility cart between the top support platform and the bottom support platform,
wherein the enclosure assembly is disposed entirely around a perimeter of the bottom support platform to define an enclosed space between the bottom support platform and the top support platform, and
wherein all fasteners used to operably couple portions of the enclosure assembly to each other are disposed inside the enclosed space.

2. The utility cart of claim 1, wherein the enclosure assembly comprises:
a first end panel disposed at a first end of the utility cart;
a second end panel disposed at a second end of the utility cart opposite from the first end;
a front panel assembly extending between, and operably coupled to, the first and second end panels and disposed at a first side of the utility cart; and
a back panel extending between, and operably coupled to, the first and second end panels and disposed at a second side of the utility cart opposite from the first side.

3. The utility cart of claim 2, wherein the first end panel comprises a first outer face, a first attachment flange, and a second attachment flange,
wherein the second end panel comprises a second outer face, a third attachment flange, and a fourth attachment flange,
wherein the first, second, third and fourth attachment flanges are disposed inside the enclosed space.

4. The utility cart of claim 3, wherein the first, second, third and fourth attachment flanges extend from the top support platform to the bottom support platform,
wherein the first, second, third and fourth attachment flanges of the first and second end panels comprise a hook disposed proximate to the bottom support platform,
wherein the hook is configured to engage a lip of the bottom support platform to operably couple the first and second end panels to the bottom support platform.

5. The utility cart of claim 3, wherein the first outer face and the second outer face are disposed in respective first and second end planes,
wherein the first, second, third and fourth attachment flanges are disposed in respective first, second, third and fourth attachment planes, and
wherein the first, second, third and fourth attachment planes are substantially perpendicular to the first and second end planes.

6. The utility cart of claim 5, wherein the back panel comprises a third outer face, a fifth attachment flange, and a sixth attachment flange, and
wherein the third outer face is disposed in a first side plane and the fifth and sixth attachment flanges are disposed in respective fifth and sixth attachment planes that are parallel to the first and third attachment planes, respectively.

7. The utility cart of claim 6, wherein the back panel further comprises a first corner portion, and a second corner portion,
wherein the first and second corner portions are shaped to extend around an exterior surface of respective ones of the plurality of upright support members,
wherein the fifth and sixth attachment flanges are disposed at the first and second corner portions, respectively, and
wherein the fifth and sixth attachment planes are parallel to the first side plane.

8. The utility cart of claim 7, wherein the first and second end panels are operably coupled to the back panel via a first fastener that operably couples the first and fifth attachment flanges to each other and a second fastener that operably couples the third and sixth attachment flanges to each other, respectively, and
wherein the first fastener is disposed perpendicular to the first and fifth attachment planes and the second fastener is disposed perpendicular to the third and sixth attachment planes.

9. The utility cart of claim 3, wherein the front panel assembly comprises:
a third corner portion, a fourth corner portion, and at least one door hingedly operably coupled to one of the third corner portion and the fourth corner portion, and
wherein the third and fourth corner portions comprise seventh and eighth attachment flanges, respectively.

10. The utility cart of claim 9, wherein the at least one door is disposed in a second side plane and the seventh and eighth attachment flanges are disposed in respective seventh and eighth attachment planes, and
wherein the seventh and eighth attachment planes are parallel to the second and fourth attachment planes, respectively.

11. The utility cart of claim 10, wherein the first and second end panels are operably coupled to the front panel assembly via a third fastener that operably couples the second and seventh attachment flanges to each other and a fourth fastener that operably couples the fourth and eighth attachment flanges to each other, respectively, and
wherein the third fastener is disposed perpendicular to the second and seventh attachment planes and the fourth fastener is disposed perpendicular to the fourth and eighth attachment planes.

12. The utility cart of claim 9, wherein the seventh and eighth attachment flanges of the front panel assembly each comprise a hook disposed proximate to the bottom support platform, and
wherein the hook is configured to engage a lip of the bottom support platform to operably couple the third and fourth corner portions to the bottom support platform.

13. The utility cart of claim 2, wherein the enclosure assembly further comprises a shelf disposed parallel to the top support platform and the bottom support platform within the enclosed space, and
wherein the shelf is supported by a support lip disposed at the first end panel, the second end panel, and the back panel.

14. The utility cart of claim 2, wherein the enclosure assembly further comprises a pattern comprising a plurality of holes that allow visibility into the enclosed space.

15. The utility cart of claim 2, wherein the enclosure assembly is opaque.

16. An accessory storage assembly for a utility cart, the accessory storage assembly comprising:
a top support platform of the utility cart;
a bottom support platform of the utility cart, the bottom support platform disposed substantially parallel to the top support platform; and
an enclosure assembly configured to be added to the utility cart between the top support platform and the bottom support platform,
wherein the enclosure assembly is disposed entirely around a perimeter of the bottom support platform to define an enclosed space between the bottom support platform and the top support platform, and
wherein all fasteners used to operably couple portions of the enclosure assembly to each other are disposed inside the enclosed space.

17. The accessory storage assembly of claim 16, wherein the enclosure assembly comprises:
a first end panel disposed at a first end of the utility cart;
a second end panel disposed at a second end of the utility cart opposite from the first end;
a front panel assembly extending between, and operably coupled to, the first and second end panels and disposed at a first side of the utility cart; and
a back panel extending between, and operably coupled to, the first and second end panels and disposed at a second side of the utility cart opposite from the first side,
wherein the first and second end panels are disposed substantially parallel to each other and substantially perpendicular to the front panel assembly and the back panel.

18. The accessory storage assembly of claim 17, wherein the enclosure assembly further comprises a shelf disposed parallel to the top support platform and the bottom support platform within the enclosed space, and
wherein the shelf is supported by a support lip disposed at the first end panel, the second end panel, and the back panel.

19. An enclosure assembly for a utility cart, the enclosure assembly comprising:
a first end panel disposed at a first end of the utility cart;
a second end panel disposed at a second end of the utility cart opposite from the first end;
a front panel assembly extending between, and operably coupled to, the first and second end panels and disposed at a first side of the utility cart; and
a back panel extending between, and operably coupled to, the first and second end panels and disposed at a second side of the utility cart opposite from the first side,
wherein the first and second end panels are disposed substantially parallel to each other and substantially perpendicular to the front panel assembly and the back panel,
wherein the enclosure assembly is disposed entirely around a perimeter of a bottom support platform of the utility cart to define an enclosed space between the bottom support platform and a top support platform of the utility cart, and
wherein all fasteners used to operably couple portions of the enclosure assembly to each other are disposed inside the enclosed space.

20. The enclosure assembly of claim 19, wherein the enclosure assembly further comprises a shelf disposed parallel to the top support platform and the bottom support platform within the enclosed space, and
wherein the shelf is supported by a support lip disposed at the first end panel, the second end panel, and the back panel.
